(19) United States
(12) Patent Application Publication
(10) Pub. No.: US 2010/0093052 A1
(43) Pub. Date: Apr. 15, 2010

(54) MAGNETIC CELL SEPARATION

(75) Inventors: Jeffrey J. Chalmers, Columbus, OH (US); Oscar R. Lara-Velasco, Bryn Mawr, PA (US); Xiaodong Tong, Columbus, OH (US); Maciej Zborowski, Bay Village, OH (US); Lee R. Moore, Sagamore Hills, OH (US); Sherif S. Farag, Carmel, IN (US)

Correspondence Address: WOOD, HERRON & EVANS, LLP, 2700 CAREW TOWER, 441 VINE STREET, CINCINNATI, OH, 45202, US

(73) Assignees: The Cleveland Clinic Foundation; The Ohio State University

(21) Appl. No.: 12/514,618

(22) PCT Filed: Nov. 14, 2007
(86) PCT No.: PCT/US07/84685

§ 371 Date: Dec. 11, 2009

Related U.S. Application Data
(60) Provisional application No. 60/865,791, filed on Nov. 14, 2006.

(51) Int. Cl. C12N 13/00; C12M 1/42

(52) U.S. Cl. 435/173.9; 435/308.1

(57) ABSTRACT

Systems and methods are provided separating a first group of cells from a mixture of at least first and second groups of cells in a suspending fluid. The system includes an annular flow channel and at least one magnetic element positioned around the exterior of the annular flow channel as to provide a radially symmetric magnetic field around at least a portion of the annular flow channel. The at least one magnetic element is configured to provide a gradually increasing magnetic field along the axis of flow as to limit the maximum magnetic gradient within the annular flow channel. A pump is operatively connected to a terminal end of the annular flow channel to force the suspending fluid through the annular channel.

### RELATED APPLICATION

The present application claims priority from U.S. Provisional Patent Application Ser. No. 60/865,791, filed Nov. 14, 2006, herein incorporated by reference in its entirety.

### FIELD OF THE INVENTION

The present invention relates to systems and methods for refining or depleting cell populations and, in particular, is directed to systems and methods for the efficient separation of targeted cells from nontargeted cells.

### BACKGROUND OF THE INVENTION

Cell sorting devices separate cell populations of interest from a suspension and/or other types of cells. The principal method of operation of early cell sorting devices relied on a cell's physical parameters to distinguish that cell from a suspension and/or other types of cells. Examples of bulk cell sorting techniques include filtration, which is based on cell size, and centrifugation, which is based on cell density. These techniques are effective as long as the cell population of interest is significantly different, with respect to size or density, from the suspension and/or the other cells in the population (e.g., a separation of red blood cells from blood.) However, when the cell population of interest does not differ significantly in size or density, filtration and centrifugation techniques are ineffectual.

As a result, immunological labeling of a cell population of interest has become a significant analytical tool in basic biological studies, applied biological studies, in the clinical diagnosis of diseases and the rapidly developing cell-based therapies in the treatment of diseases. The ability to analyze and separate a heterogeneous cell population on the basis of cellular properties/characteristics is a significant analytical and preparative resource. Essentially, immunological tagging allows for the differentiation of cells based upon the presence of specific surface receptors to which specific antibodies bind. Typically, a population of antibodies can be covalently linked to a molecule, a particle, or a support matrix to impart the cell with a desired property. In the case of immunomagnetic labels, the imparted property is a high magnetic susceptibility of the cell-label complex. Subsequently, a magnetic field can be used to quickly separate such cells based on their magnetic susceptibility.

Magnetic cell separation technology can be categorized into several different modes of operation with respect to how targeted cells are removed from a cell suspension of targeted and nontargeted cells. A batch mode of operation consists of placing a cell suspension of targeted cells within a magnetic field, and then decanting the nontargeted cells still in the cell suspension. A semi-batch mode operation consists of retaining the targeted cells within the separation device and allowing the nontargeted cells to flow through the device. A completely flow through mode of operation consists of a cell suspension of targeted and nontargeted cells entering the cell separation device and at least two exit streams leaving the device consisting of one stream containing non-targeted cells and the other exit stream containing the targeted cells.

### SUMMARY OF THE INVENTION

In accordance with an aspect of the present invention, a method is provided for separating a first group of cells from a mixture of at least first and second groups of cells in a suspension. The cells in the first group are magnetically labeled to produce a plurality of targeted cells having a selected magnetic susceptibility. An optimal volumetric flow for the mixture is determined from the lowest magnetic susceptibility of the plurality of targeted cells. The suspension is pumped through a magnetic field at the determined optimal volumetric flow rate, as to maximize a percentage of the first group of cells that are magnetically attracted to the surface through which the magnetic field is creating an attractive force.

In accordance with an aspect of the present invention, a cell separator system is provided that separates a first group of cells from a mixture of at least first and second groups of cells in a suspending fluid. The system can include an annular flow channel and at least one magnetic element positioned around the exterior of the flow channel as to provide as symmetric magnetic field around at least a portion of the flow channel as possible. The at least one magnetic element is configured to provide a gradually increasing magnetic field along the axis of flow as to limit the maximum magnetic gradient within the annular flow channel. A pump is operatively connected to a terminal end of the annular flow channel to force the suspending fluid through the annular channel.

In accordance with yet another aspect of the present invention, a system is provided that separates a first group of cells from a mixture of at least two groups of cells in a suspending fluid. The system includes an annular flow channel and at least one magnetic element substantially encompassing the annular flow channel, such that a magnetic field is provided through at least a selected portion of the annular flow channel. A pump is operatively connected to a terminal end of the annular flow channel, to force the suspending fluid through the annular channel at a specific flow rate. The flow rate and the magnitude of the magnetic field are selected to maintain a ratio of magnetic pressure created by the magnetic field to a shear stress generated by the pump within a desired range.

In accordance with yet another aspect of the present invention, a system is provided that separates a first group of cells from a mixture of at least first and second groups of cells in a suspension fluid such that the laminar shear stress on the solid surfaces of the system is at or above a given hydrodynamic shear stress such that it prevents any non-specific binding of cells to any surface, except the surface by which the cells are held due to the magnetic force.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a simplified representation of a cell separator system in accordance with an aspect of the present invention;

FIG. 2A illustrates a chart of the log₁₀ depletion of a targeted cells (z-axis) from passage through a magnetic cell separator as a function of hydrodynamic shear stress (x-axis) and magnetic pressure (y-axis);

FIG. 2B illustrates a chart of the fractional recovery (z-axis) of an nontargeted cell population from a magnetic cell separator as a function of hydrodynamic shear stress (x-axis) and magnetic pressure (y-axis);

FIG. 3A illustrates a plot of the recovery of nontargeted cells, as a function of hydrodynamic shear stress;

FIG. 3B illustrates a plot of the log₁₀ depletion of targeted cells as a function of the ratio of the magnetic pressure to shear stress;

FIG. 4 illustrates an exemplary implementation of a cell separation system in accordance with an aspect of the present invention;

FIG. 5A illustrates a portion of an exemplary design for one type of a magnet assembly in accordance with an aspect of the present invention;

FIG. 5B is a plot of the magnetic field (y-axis) in units of tesla, produced by the magnet assembly of FIG. 5A applied along the length of a flow channel (x-axis) in units of millimeters from the center of the magnet assembly;

FIG. 6A illustrates a portion of a second exemplary design for a second type of magnet assembly in accordance with an aspect of the present invention;

FIG. 6B is a plot of the magnetic field, (y-axis) in units of tesla, produced by the magnetic assembly of FIG. 6A applied along the length of a flow channel (x-axis) in units of millimeters from the center of the magnet assembly;

FIG. 7A illustrates a top down view of one implementation of a magnet assembly positioned around an associated flow channel in accordance with an aspect of the present invention;

FIG. 7B illustrates a chart of the magnetic field (y-axis), in units of tesla, applied directionally around an annular flow channel, in units of angular degree, encompassed by the magnet assembly illustrated in FIG. 7A; and

FIG. 8 illustrates a method for separating a first group of cells from a mixture of at least first and second groups of cells in a suspension in accordance with an aspect of the present invention.

### DETAILED DESCRIPTION OF THE INVENTION

In accordance with an aspect of the present invention, a magnetic cell separation system is described herein that is configured to maintain optimal ranges of fluid mechanical forces and magnetic forces within each portion of the cell separation system. This design achieves a high level of performance with respect to nearly complete recovery of nontargeted cells and a high level of recovery or depletion of magnetically targeted cells from a heterogeneous cell population. For the purposes of this description, a “targeted” cell is a cell that is intended to be retained by the magnetic cell separation system. A targeted cell population will have magnetic properties that differentiate it from one or more groups of untargeted cells. These magnetic properties can arise, for example, as the result of any of a number of affinity based techniques, (i.e., antibody-magnetic particle conjugates or ligand-magnetic particle conjugates), specific manipulation of cells that make them magnetic, (i.e., the deoxygenation of red blood cells which renders the cell magnetic), or simply represent an innate property of the cells.

Embodiments of the current disclosure may be used in the biomedical, biotechnological, pharmaceutical, and chemical industries, and may be used for purifications, including, but not limited to cell enrichment and selection. When magnetic cell separations are used in a clinical setting, they may be useful for diagnostic assays such as cancer screening, cancer monitoring, as a preparatory tool for therapeutic techniques such as for transplantation, and diabetes and stem cell therapies.

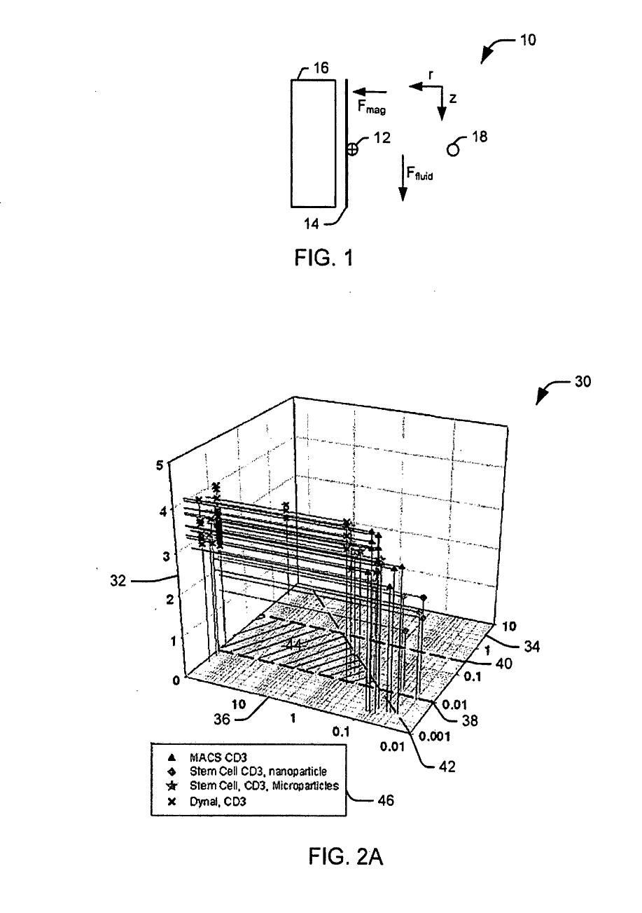

FIG. 1 illustrates a simplified representation of a cell separator system 10 in accordance with an aspect of the present invention. In the illustrated system, a targeted cell 12 is attracted to a wall 14 of a flow channel via a magnetic field, H, provided by a magnetic pole piece 16. Along with attractive force of the magnet 16, the cell 12 is acted upon by a hydrodynamic force, F_{fluid}, produced by a pressure difference. Typically, this pressure difference can be produced by a pump, but other methods are possible. A second, nontargeted, cell 18 is acted upon solely by the hydrodynamic force. In an ideal system, the magnetic force will be sufficient to trap each targeted cell 12 while the hydrodynamic force will be sufficient to prevent all of the nontargeted cells 18 from adhering to any surface in the separator, including the channel wall 14 or among cells (e.g., 12) already held on the wall. To this end, a cell separator system in accordance with an aspect of the present invention is described herein that is configured to maintain the magnetic and hydrodynamic forces at optimal levels to provide for efficient and accurate separation of populations of cells.

### Magnetic Pressure

At the most fundamental level, a magnetic force, F_{mag,p}, acting on a magnetic particle bound to a cell is:

F_{mag,p}=MₚVₚ∇B₀ Eq. 1

where Mₚ is the particle volume magnetization, Vₚ is the particle volume, B₀ is the applied magnetic field and ∇ is the gradient operator (or so called nabla operator). The magnetic field interacts with the attached beads creating a stress on the cell surface. Here it is assumed that the particle magnetic moment is aligned with the applied magnetic field. For the purpose of this analysis, it is assumed that the beads attach to the cell with a uniform surface number density σ, and that the mechanical stress is a continuous function of the surface coordinates due to the averaging effects of the cell cytoskeleton. It will be appreciated that while this analysis focuses on immunomagnetically labeled cells, the analysis can be easily extended to cases in which the magnetic properties of the cells arise from other mechanisms.

For cells containing naturally occurring magnetic species, such as deoxy-hemoglobin in the erythrocytes or magnetite crystals in the magnetotactic bacteria, the expression for the magnetic force acting on cell is modified to include the magnetic moment contributions from those species. Typically, the magnetization of the fluid medium (aqueous solution of electrolytes at physiologic concentrations) is much lower than that of the magnetically labeled cell, and therefore is not addressed in this current analysis. In the presence of the magnetic species in solution, such as gadolinium ions or ferrofluids used as MRI contrast agents, the analysis can be extended in a straightforward manner. The simplifying assumptions used here still allow capturing the key features of the system under consideration.

Assume a cell deposited on a solid wall (or held against a solid wall by a magnetic force) as shown in FIG. 1, with a magnetic force, F_{mag}, directed perpendicularly to the deposition wall. The effect of the magnetic force is equivalent to the action of a pressure that pushes the cell against the wall. This pressure is associated with the action of the magnetic labels, attached to the cell, and their interaction with the magnetic field. In analogy to the magnetic-fluid pressure, defined in ferrohydrodynamics, and the magnetostatic field pressure, defined in magnetohydrodynamics, we define the magnetic pressure, associated with the action of the magnetic field on the labeled cells, by the following formula:

$${p}_{mag} ≡ {χ}_{eff} \frac{{B}_{0}^{2}}{2 {μ}_{0}} \qquad Eq . 2$$

where χ_{eff} is the effective magnetic susceptibility of the cell-label complex and μ₀=4π×10⁻⁷ tesla·ampere/meter (T·A/m) is a constant. The magnetic susceptibility is a unit-less quantity. The unit of the magnetic field intensity in International System of Units (SI), tesla, is equivalent to the units of newton/(A·m), which, when inserted into Eq. 2 together with the unit of μ₀, result in the unit of pressure, N/m², as it should be.

Thus, the mechanical pressure of the magnetic beads bound to the cell surface results in a pressure exerted on the cell as a whole that pushes against the channel wall 14. An explicit relationship between the magnetic force, F_{mag}, bringing the cell in contact with the channel wall 14, and the associated magnetic pressure, p_{mag}, acting on the cell (Eq. 2), is obtained by noting that the following relationship holds between the magnetic force density, f_{mag}, and the magnetic pressure gradient:

f_{mag}=∇p_{mag} Eq. 3

where ∇ denotes the gradient operator.

The total force acting on the cell, F_{mag}, is equal the product of the force density times the volume, V, of the cell-label complex, so that:

F_{mag}=f_{mag}V Eq. 4

Thus, by combining Eqs. 3 and 4, one obtains the desired relationship between the magnetic force and the magnetic pressure exerted on the cell due to the combined action of the magnetic labels attached to the cell:

F_{mag}=V∇p_{mag} Eq. 5

where p_{mag} is a function of the applied magnetic field and the effective magnetic susceptibility of the cell-label complex, as expressed in Eq. 2.

Note that the magnetic force acting on the cell is directly proportional to the magnetic pressure gradient rather than the magnetic pressure itself. This is analogous to the relationship between the force densities and pressures in fluid dynamics (with an important distinction that the magnetic force density acts in the direction of the increasing magnetic pressure, Eq. 5, while that of the viscous fluid acts in the direction of the decreasing fluid pressure). An important consequence of this fact is that the magnetic force on the cell vanishes in the region of a constant magnetic field. Conversely, in order to increase magnetic forces acting on the labeled cell, one seeks to increase the magnetic pressure gradient. This is achieved by designing magnets that produce high magnetic fields and gradients (compare Eqs. 2 and 5) and by using highly magnetic labels that impart high effective magnetic susceptibility, χ_{eff}, to the cell-label complex (Eq. 2).

Another important note regarding the magnetic pressure formula in Eq. 2 is that it applies only to the type of magnetic labels for which the effective cell-label complex susceptibility, χ_{eff}, is independent of the applied field, H (such as in the case of superparamagnetic nano- and micro-particles). If the magnetic label magnetization, Mₚ, saturates in the applied field, such as in the case of ferromagnetic particles, the magnetic pressure acting on a single label is:

p_{mag,p}=MₚB₀ Eq. 6

where Mₚ is independent of the applied field H.

Note that upon substitution of the above expression into Eq. 5 one recovers the original formula for the force on the magnetic label, shown in Eq. 1, providing that the volume of the magnetic material, V, is equal to the volume of the single label particle, Vₚ, as it should. For the volume of the magnetic material that is a sum of volumes of the magnetic labels attached to the cell one obtains:

V=NVₚ Eq. 7

where N is the number of label particles attached to the cell.

The insertion of Eqs. 7 and 6 into Eq. 4 leads to the following expression of the magnetic force acting on the cell:

F_{mag}=NVₚMₚ∇B₀=NF_{mag,p} Eq. 8

where the last equality results from using Eq. 1. In other words, the magnetic force acting on the cell is a sum of the magnetic forces acting on a single label particle. This illustrates a simple fact that the magnetic force acting on the cell, depicted in FIG. 1, increases with the increasing number of magnetic labels attached to the cell.

The above analysis was presented to bring out the essential features of the magnetic pressure in application to the magnetic separation and therefore was based on a number of simplifying assumptions. For instance, Equation 8 strictly applies only if the magnetic moments, MₚVₚ, of all the attached particles are aligned with the applied field, H. This may not necessarily be the case because of the randomizing effects of the particle binding outside the magnetic field, the resistance to torsion of the cell structures involved in the magnetic particle binding, and the superparamagnetic behavior of the single domain nanoparticles.

In summary, the mechanical force exerted on a cell surface by the magnetic particles attached to that surface is directly proportional to the magnetic pressure gradient. In particular, for constant gradient magnetic fields, the magnetic force is directly proportional to the difference in the magnetic field pressure across the cell.

### Shear Stress

Turning to the forces applied by the medium, three types of parameters are of particular interest for characterizing the effect of hydrodynamic forces, on cells: shear stresses, normal stresses, and the energy dissipation rate. For well designed and operated cell separation systems, it is possible to both know and control the hydrodynamic shear stress that the flowing cell suspension exerts on all of the surfaces of the system. At the most basic level, shear stress is mathematically defined as;

SS=ηγ Eq. 9

where SS is the shear stress (SI units are N/m², same as those of the pressure), η is the viscosity of the fluid, and γ is the shear rate.

For laminar flow, the maximum shear stress typically occurs at the walls, and for simple geometries, this maximum shear stress can be calculated analytically. For example, for two parallel plates, the shear stress of the fluid flow operating on the surface of the plates is defined by:

$$SS = \frac{6 η Q}{{wh}^{2}} \qquad Eq . 10$$

where η is the fluid viscosity, Q is the volumetric flow rate, w is the width of the plates, and h is distance between the plates.

For a pipe, the wall shear stress is defined by:

$$SS = \frac{4 η Q}{π {R}^{3}} \qquad Eq . 11$$

For either the inner or outer wall of an annulus, the shear stress is:

$$SS = η ( \frac{4 Q}{( 1 - {κ}^{4} - \frac{{( 1 - {κ}^{2} )}^{2}}{ln ( \frac{1}{κ} )} ) π {R}^{3}} ( \frac{r}{R} - \frac{1 - {κ}^{2}}{ln ( \frac{1}{κ} )} \frac{R}{r} ) ) ; κ = {R}_{inner} / R \qquad Eq . 12$$

where Rᵢₙₙₑᵣ is the inner radius of the annulus and R is the radius of the outer wall.

A second type of fluid stress, referred to as normal stress, typically occurs in non-wall regions of flow. Normal stresses, which typically occur when a contraction of the fluid flow occurs, have been strongly suggested to have a negative impact on cells. In a cell separation system, such geometries exist in a nozzle of a syringe pump or in a tubing connector which connects two tubes together.

A third parameter, which is often used as an alternative to the use of shear or normal stress, is the local energy dissipation rate (EDR). The EDR, is a scalar value with units of power per unit volume (e.g., W/m³) that represents the irreversible rate of internal energy increase per unit volume, or the irreversible conversion of mechanical energy to heat. Fundamentally, the EDR, denoted here as ε, can be determined using the following equation:

ε=τ:∇U Eq. 13

where τ is the stress tensor and ∇U is the velocity gradient tensor, defined as:

$$∇ U = [ \begin{matrix}\frac{\partial {U}_{x}}{\partial x} & \frac{\partial {U}_{y}}{\partial x} & \frac{\partial {U}_{z}}{\partial x} \\ \frac{\partial {U}_{x}}{\partial y} & \frac{\partial {U}_{y}}{\partial y} & \frac{\partial {U}_{z}}{\partial y} \\ \frac{\partial {U}_{x}}{\partial z} & \frac{\partial {U}_{y}}{\partial z} & \frac{\partial {U}_{z}}{\partial z}\end{matrix} ] \qquad Eq . 14$$

For an incompressible Newtonian fluid, the EDR can be expressed as:

ε=η(∇U+∇U^{T}):∇U Eq. 15

where ∇U^{T} is the transpose of ∇U.

Alternatively, the EDR can be expressed in terms of the second invariant of the rate of deformation tensor, 2D:

ε=−μII_{2D} Eq. 16

The second invariant and the rate of deformation are defined as:

$${Π}_{2 D} = \frac{1}{2} [ {( tr 2 D )}^{2} - {tr ( 2 D )}^{2} ] \\ tr 2 D = ∇ U + ∇ {U}^{T} \qquad Eq . 17$$

where tr2D is the trace of the rate of deformation tensor.

As shown above, once the velocity vectors are known at every point in a given flow system, the energy dissipation rate at every point (the local EDR) can be determined. Also, for a laminar, Newtonian flow, which is typical for a magnetic cell separation system, a value for shear stress can easily be converted to an EDR.

It will be appreciated that a significant amount of hydrodynamic force is useful in a cell separation system, not only for moving the cell suspension through the device, but also for the shear stress created by the flow on the various surfaces in the system that can prevent non-specific attachment of cells to these surfaces. Unfortunately, high levels of hydrodynamic forces can have significant, deleterious effects. For many cell lines, such as CHO-K1, damage to cells can occur at levels of EDR of approximately 10⁶ W/m³ for cells in suspension, and 10³ W/m³ for cells attached to surfaces. This value of EDR corresponds to a value of shear stress of approximately 50 N/m² on a solid surface. It will be appreciated, however, that the sensitivity of cells to damage from applied hydrodynamic force varies across cell lines, and that even within a given cell line, factors such as the method of growth of the cells (anchoraged, suspended, etc.) can influence the tolerance of the cells for high levels of fluid pressure.

Even where the hydrodynamic forces are insufficient to damage the cells, high levels of hydrodynamic force can break cell-antibody bonds. For example, recent research has determined that it takes 256 pN at a pulling velocity of 166.7 nm/s to break a human IgG1 and anti-human IgG1 pair. Given that the cross sectional area of a human lymphocyte is 3.85×10⁻¹¹ m², using this value of 256 pN, the pressure required to break a single antibody-antigen interaction between a cell and magnetic particle is 6.7 N/m². For removing a cell bound to a surface through specific cell-antibody binding, reported values for the necessary shear stress have ranged from 0.05 N/m² to over 50 N/m². It is highly likely that this range is at least partially representative of the specific number of antibody-antigen binding events per attached cell.

Accordingly, excessive levels of EDR and SS have been found to have inhibitory effects on magnetic cell separation. For example, levels of EDR of 8 kW/m³ (or a SS of 2.8 N/m²) on the wall of the magnetic deposition zone in one implementation of the cell separator resulted in cell-Dynal bead movement down the length of the magnetic separation zone as a result of flow and a significantly lower depletion of unwanted cells when compared to identical operation with a wall EDR of 4×10⁻³ kW/m³ (or a SS of 1.7×10⁻³ N/m²). It will be appreciated that 2.8 N/m² is well below the range of shear stress known to be lethal to cells. This critical value of approximately 2.8 N/m² holds true even for the narrower holes of the flow distribution manifold, which can have a diameter of 500 microns, for example. These effects can be exacerbated inside of a syringe pump, as regions of localized hydrodynamic force within the pump can be sufficient to remove the magnetic label from the cell. Accordingly, in accordance with an aspect of the present invention, the rate of fluid flow within the flow channel can be regulated such that the maximum value of the local energy dissipation rate anywhere within the flow channel does not exceed five hundred W/m³.

It will be appreciated, however, that the effects of an insufficient hydromechanical force can be equally problematic. Without sufficient fluid pressure, non-tagged cells can be trapped against the walls along with the magnetically tagged cells, thereby reducing the yield of the system. To this end, in accordance with an aspect of the present invention, an optimal flow range has been determined that is sufficient to clear non-labeled cells from the system (both in the magnetic deposition and non-deposition zone), while avoiding cell death or separation of the labels from the tagged cells.

In accordance with an aspect of the present invention, a shear-induced diffusion mechanism, that is, the flow-dependent migration of cells away from the walls of the separator, is precisely controlled to regulate the cake formation of magnetically captured cells on the wall of the flow channel. By maintaining precise control over the distribution of the magnetically-tagged and un-tagged cells between the cake and the flowing suspension, the purity of the cake and the recovery of the wanted cells can be enhanced, and the performance of the magnetic separation process can be improved over existing magnetic separator designs. In particular, it prevents non-magnetic, non-specific adhesion of cells to the channel inner surfaces, thus improving recovery (also known as the yield) of the un-tagged cell fraction.

For a tube (pipe) flow, this optimum range of shear stresses, assuming all quantities in SI units, corresponds to the following mathematical conditions:

$$0.01 \frac{N}{{m}^{2}} \leq \frac{4 η Q}{ π {R}^{3}} \leq 0.5 \frac{N}{{m}^{2}} \qquad Eq . 18$$

For flow in an annulus, this optimum range of shear stresses corresponds to the following mathematical conditions:

$$0.01 \frac{N}{{m}^{2}} \leq η ( \frac{4 Q}{( 1 - {κ}^{4} - \frac{{( 1 - {κ}^{2} )}^{2}}{ln ( \frac{1}{κ} )} ) π {R}^{3}} ( \frac{r}{R} - \frac{1 - {κ}^{2}}{2 ln ( \frac{1}{κ} )} \frac{R}{r} ) ) \leq 0.5 \frac{N}{{m}^{2}} \qquad Eq . 19$$

Accordingly, the acceptable range of volumetric flow rate, with all units in the SI system, for a given system is constrained by the geometry of the system, such that for a tube:

$$\frac{0.0025 N}{{m}^{2}} \frac{π {R}^{3}}{η} \leq Q \leq \frac{0.125 N}{{m}^{2}} \frac{π {R}^{3}}{η} \qquad Eq . 20$$

and for an annulus:

$$\frac{\frac{0.0025 N}{{m}^{2}} ( 1 - {κ}^{4} - \frac{{( 1 - {κ}^{2} )}^{2}}{ln ( \frac{1}{κ} )} ) π {R}^{3}}{η ( \frac{r}{R} - \frac{1 - {κ}^{2}}{2 ln ( \frac{1}{κ} )} \frac{R}{r} )} \leq Q \leq \frac{0.125 \frac{N}{{m}^{2}} ( 1 - {κ}^{4} - \frac{{( 1 - {κ}^{2} )}^{2}}{ln ( \frac{1}{κ} )} ) π {R}^{3}}{η ( \frac{r}{R} - \frac{1 - {κ}^{2}}{2 ln ( \frac{1}{κ} )} \frac{R}{r} )} \qquad Eq . 21$$

It will be appreciated that a functional cell separator may have fluid passing through channels of various sizes and shapes. For example, one implementation of the present invention utilizes an annular flow channel and a tubular exit line. Operation of the system with flow rates bounded as set forth in Equations 20 and 21 creates shear stress on surfaces within the device such that non-specific attachment of cells to the walls of the tube and annulus is prevented. This is in contrast to all other known magnetic cell separation designs, both commercial and research. The present invention utilizes specific dimensions for all parts that come in contact with the cell suspension, including tubing, tubing fitting, and the region in which cells are magnetically deposited, such that the cell separator can be operated to maintain the entire flow path of the cells at the desired shear stress levels.

It will further be appreciated that the magnetic force capturing the labeled cells must be sufficient to hold the cells in place for a given flow rate. To this end, a non-dimensional factor, ζ, has been defined to predict the performance of a cell sorted when operated in the deposition mode of operation. This ζ factor is defined as the ratio of two dynamic terms representing the hydromechanical force and the magnetic force, namely a maximum fluid flow velocity, vₘₐₓ, and a maximum magnetically induced velocity, uₘₐₓ, such that:

$$ζ = \frac{{u}_{max}}{{v}_{max}} = ( \frac{2}{9} ) \frac{{R}^{2}}{η {v}_{max}} \frac{Δ χ}{{μ}_{0}} \frac{{B}_{0}^{2}}{{r}_{o}} \\ where {S}_{m} = \frac{d}{d z} ( \frac{{B}_{0}^{2}}{2 {μ}_{0}} ) \qquad Eq . 22$$

is the magnetic driving force (equal to the magnetic pressure gradient in free space). The definition of the ζ factor is based on the simplest case of the magnetic field pressure being a linear function of the spatial coordinates, so that

$${S}_{m} = \frac{1}{2 {μ}_{0}} \frac{{B}_{0}^{2}}{{r}_{0}} ,$$

where r₀ is a characteristic distance (such as the radius of the aperture of a magnetic element in the quadrupole field system) along which the field increases from 0 to B₀.

Alternatively, another analysis can be made based on the static ratio of the magnetic pressure induced on cells deposited within the separator and the fluid shear stress acting on these deposited cells, perpendicular to the magnetic force. This static ratio can be used to optimize the performance of a given cell separation system. It will be appreciated that both the magnetic pressure and the shear stress can be expressed in units of N/m², which allows straightforward comparison of the two pressures. As discussed previously (See, e.g., Equations 22-25 and the accompanying discussion), the shear stress is a function of the geometry of the system and the volumetric flow rate.

For comparison, the numerical value of the mechanical pressure exerted on a cell by the bound magnetic particles due to their interaction with the magnetic field, or the magnetic pressure, p_{mag}, is calculated. For the magnetic field of B₀=1 T, representative of the magnetic cell separation applications, the numerical value of the term

$$\frac{{B}_{0}^{2}}{2 {μ}_{0}}$$

entering the equation for the magnetic pressure (Eq. 2) is approximately equal to 4×10⁵ N/m². For a magnetic field gradient of

$$100 \frac{T}{m} ,$$

and a spatial separation of 10 μm=10⁻⁵ m (or an average cell diameter), which is representative of magnetic cell separation applications, the magnetic field difference is 10⁻³ T across the cell diameter. Further, the effective susceptibility of the magnetically labeled cell is typically in the range of χ_{eff}≈10⁻⁴ to 10⁻², resulting in the magnetic pressure difference across the cell on the order of p_{mag}≈0.1 to 0 N/m², correspondingly (Eq. 2).

This magnetic pressure difference across the cell linear dimension is the agent that pushes the cell against the channel wall causing it to separate from the unlabeled cells, as discussed in the previous section. In the following analysis it is referred to simply as “the magnetic pressure”, for short. In that context, the magnetic pressure is to be understood as measured at the side of the cell that is adjacent to the channel wall relative to the pressure that is measured at the side of the cell that is away from the channel wall. Consequently, we adopt a convention by which the magnetic pressure is measured with respect to a reference point that is located on the distal part of the cell surface relative to the channel wall. By that convention, the magnetic pressure is zero when it does not change across the cell width. The range of the magnetic pressure may extend to 100 N/m² for higher fields and higher gradients. For example, up to approximately 2 T and 1,000 T/m can be achieved using current permanent magnet technology.

In accordance with an aspect of the present invention, the ratio of magnetic pressure to fluid shear stress can also be controlled for the nontargeted cells, to ensure that any magnetic attraction of the nontargeted cells to the wall of the cell separator is effectively countered by the fluid pressure. Ideally, non-targeted cells should have no magnetic susceptibility (most cells are diamagnetic). However, various levels of non-specific binding of binding of magnetic labeling particles can occur.

In accordance with an aspect of the present invention, the ratio of the magnetic pressure on the nontargeted cells to the fluid shear stress is maintained below 0.1. To this end, a magnetic labeling process can be controlled to ensure that the targeted cells have a significantly higher effective magnetic susceptibility than the nontargeted cells. This can be accomplished in a number of ways, including the use of high quality magnetic particles that have little to no nonspecific binding or magnetic particles that are sufficiently large that the hydrodynamic forces operating on the magnetic particle to remove it from the cells is higher than the non-specific forces holding the cell magnetic particle together. For example, large (e.g., on the order of sixty to one hundred eighty nanometers in diameter) nanoparticles with significant iron content conjugated to specific antibodies can be used to inhibit nonspecific binding of the particles. Other desirable particle properties include chemical composition and surface geometry (smooth, rugged) that minimize or eliminate any or all of the following phenomena: physical particle adhesion to cell surface, particle phagocytosis, endocytosis or pinocytosis, particle recognition by cell surface receptors and the resulting cell activation and intracellular signaling.

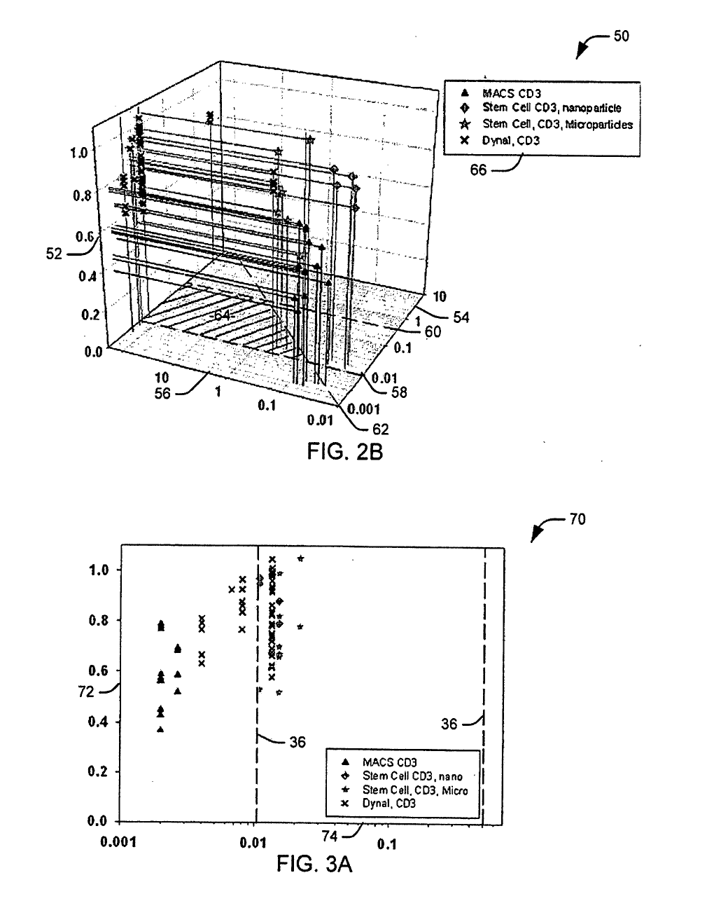

FIGS. 2A and 2B are chart 30 and 50 illustrating two primary measures of magnetic separator performance: the depletion of magnetically targeted cells, presented on a logarithmic scale, and the recovery of the nontargeted cell population, both as a function of the hydrodynamic shear stress and magnetic pressure on the deposition surface in accordance with an aspect of the present invention. Each chart 30 and 50 contains results for a number of experimental runs in which the CD3 cell surface markers on human blood were immunomagnetically labeled and magnetically separated with the specific magnetic pressure and shear stress. The different symbols correspond to four different commercial immunomagnetic reagents, as indicated in the respective legends 46 and 66 of the charts.

FIG. 2A illustrates a chart 30 of the depletion of targeted cells from passage through a magnetic cell separator. The depletion of the targeted cells is represented on a z-axis 32 on a base-10 logarithmic scale as a function of hydrodynamic shear stress, represented in newtons (N) per square meter (m²) on an x-axis 34, and magnetic pressure, represented in newtons per square meter (N/m²) on a y-axis 36. As discussed previously, a magnetic cell separator in accordance with an aspect of the present invention is designed to operate within an optimum range in shear stress. A first boundary line 38 represents a minimum value for the shear stress that prevents nonspecific bonding of cells to the deposition surface. A second boundary line 40 represents a maximum value above which inhibitive, hydrodynamic forces can occur.

As is demonstrated by FIG. 2A, as the ratio of the magnetic pressure to shear stress increases, the log depletion of the targeted cells increases. In accordance with an aspect of the present invention, it has been determined that a ratio of magnetic pressure to fluid shear stress of at least ten is suggested to prevent the removal of magnetically deposited cells, thereby increasing the log depletion. A third boundary line 42 represents this ratio. Optimum magnetic cell separation can be achieved when the system is operated such that the shear stress is between 0.01 and 0.5 N/m², and that the magnetic pressure is at least 10 times higher than the wall shear stress. This zone of optimal operation 44 is presented in FIG. 2A as the region contained within the boundary lines 38, 40, and 42 and highlighted by diagonal lines.

FIG. 2B illustrates a chart 50 of the fractional recovery of an nontargeted cell population from a magnetic cell separator, represented as a ratio of recovered nontargeted cells to total nontargeted cells on a z-axis 52, as a function of hydrodynamic shear stress, represented in N/m² on an x-axis 58, and magnetic pressure, represented in N/m² on a y-axis 58. First and second boundary lines 58 and 60 represent a range of values for the shear stress that prevents nonspecific bonding of cells to the deposition surface, while avoiding inhibitive, hydrodynamic forces. A third boundary line 62 represents a ratio of magnetic pressure to fluid shear stress of at least ten, which, in accordance with an aspect of the present invention, has been found to prevent the removal of magnetically deposited cells while allowing nontargeted cells to be removed from the wall by fluid shear stress, thereby enhancing the recovery of nontargeted cells. A zone of optimal operation 64 defined by these parameters is presented in FIG. 2B as the region contained within the three boundary lines 58, 60, and 62.

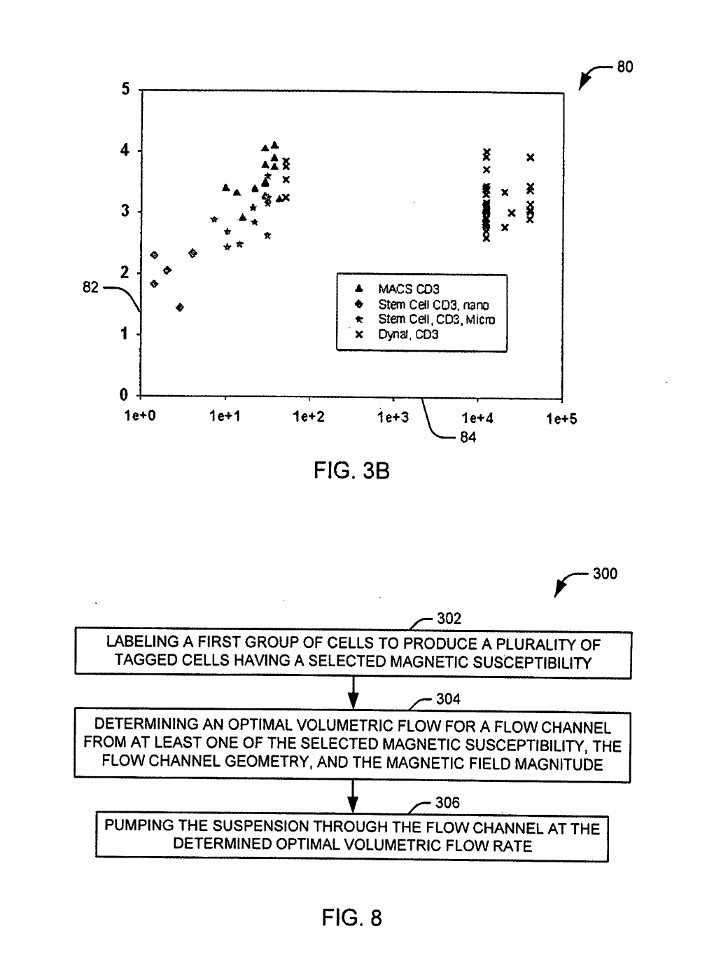

FIG. 3A illustrates a two-dimensional plot 70 of the recovery of nontargeted cells, represented on a y-axis 72 as a ratio of recovered nontargeted cells to total nontargeted cells, as a function of hydrodynamic shear stress, represented on an x-axis 74 in units of N/m². A first boundary line 36 represents a minimum value, at 0.01 N/m², for the shear stress that prevents nonspecific bonding of cells to the deposition surface. A second boundary line 36 represents a maximum value, at 0.5 N/m², above which inhibitive, hydrodynamic forces can occur. As FIG. 3A demonstrates, the recovery of nontargeted cells is greatest within the range between 0.01 N/m² to 0.5 N/m².

FIG. 3B illustrates a two-dimensional plot 80 of the depletion of targeted cells, represented on a y-axis 82 on a base-10 logarithmic scale, as a function of the ratio of the magnetic pressure to shear stress, represented on an x-axis 84. As FIG. 3B illustrates, as the ratio of the magnetic pressure to shear stress increases, the log depletion of the targeted cells increases. It will be appreciated from the illustrated data that a ratio of magnetic pressure to fluid shear stress of at least ten is suggested to prevent the removal of magnetically deposited cells, thereby increasing the log depletion.

It will be appreciated that the magnetic pressure and the magnetic gradient at each point in the flow channel is a factor of the strength and configuration of a magnetic element in the device, and can be manipulated within certain practical limits. Similarly, the shear stress and the maximum fluid velocity are functions of the geometry of the device and the volumetric flow rate, both of which can be selected within practical limits. Accordingly, through careful selection of these parameters, a desired value of shear stress and/or ratio at all points of the system can be maintained, allowing for optimal system performance.

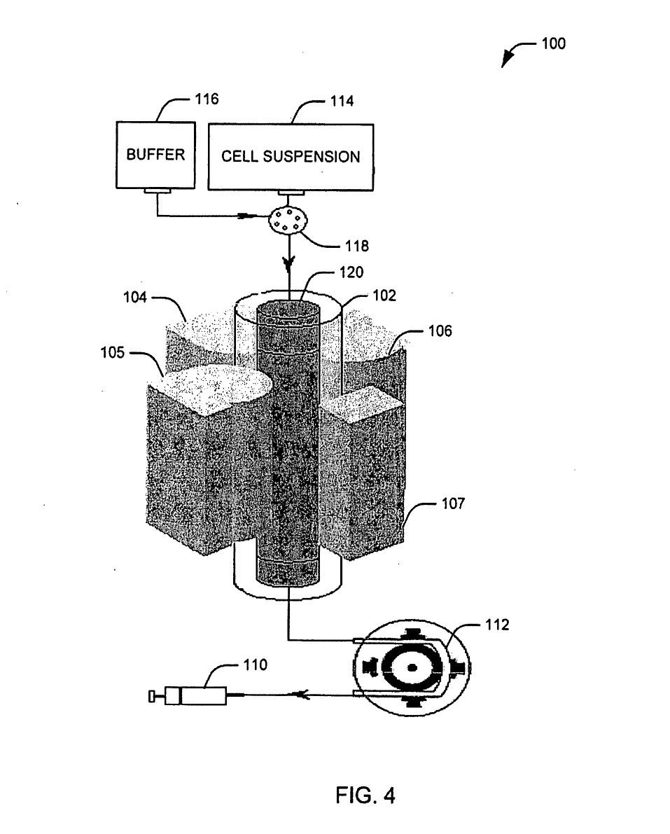

FIG. 4 illustrates an exemplary implementation of a cell separation system 100 in accordance with an aspect of the present invention. The cell separation system 100 is optimized to maintain magnetic and hydrodynamic forces within optimal ranges as to maximize the yield of a targeted cell population. The illustrated system 100 is a semi-batch system, in which a targeted cell population is held to the outer wall of a flow channel 102 via a magnetic field provided by four magnetic pole pieces 104-107 while the remaining cells and fluid are removed by an associated pump 110 through an exit line 112. It will be appreciated, however, that the configuration of the cell separation system 100 can be varied according to a number of factors, including the type of cells being sorted, the fluid medium, the method by which the fluid is transported through the system, and the type of container used for collecting the sorted cell sample.

During operation, a user can release a cell suspension 114, containing a mixture of targeted cells and nontargeted cells, and a buffer fluid 116 from associated containers by opening a valve 118 at the opening to the annular flow channel 102. The pump 110 can be activated to draw the cell suspension and buffer fluid through the flow channel at a desired flow rate. It will be appreciated that the pump is located at the exit line 112 after the flow channel 102 to prevent potential removal of magnetic label, where the targeted cell population includes magnetically labeled cells, from a target cell by local regions of high hydrodynamic forces within the pump.

As the fluid passes through the flow channel 102, the targeted cells are drawn to the outer surface of the flow channel and held in place by a magnetic force. The nontargeted cells and the fluid continue through the flow channel 102 to the exit line 112, where they can be extracted from the system. In the illustrated example, each of the flow channel 102, the containers for the buffer fluid 116 and the cell suspension 114, the sorted cell fraction container within the pump 110, and the exit line 112 can be made disposable to allow for easy removal of the separated cells from the system 100 and to reduce the possibility of contamination of the system between batches of cells.

The illustrated system provides several advantages over previous cell separation systems. For example, it will be appreciated that the magnetic fields provided by the four magnetic pole pieces 104-107 will substantially cancel one another out along an axis equidistant from the four pole pieces. Accordingly, along that center axis will be a “dead zone” in which the magnetic pressure on any targeted particles would be substantially attenuated. In the illustrated system 100, the flow channel 102 is annular, with a solid core 120 in the center of the annulus. Accordingly, the suspending fluid, while between the pole pieces, is directed to the regions in which a substantial, well defined, magnetic field can be applied to the targeted particles, increasing the yield of the cells of interest. This allows the magnetic fields to be applied in a substantially uniform manner without placing magnetic structures in a manner that would obstruct the path to the flow channel 102, either directly by introducing a magnetic element into the flow path, or indirectly, by producing localized regions of high magnetic field gradient that attract large groups of targeted cells. Such obstructions can cause clumping of the cells at the obstruction, reducing the efficiency of the system by obstructing the fluid flow and reducing the yield of the separator by providing a location in which nontargeted cells can accumulate.

For example, in a high-gradient magnetic separation (HGMS) system, sub-millimeter ferromagnetic microspheres are used to generate intense local fields and gradients at the microsphere surfaces. The maximum fields are on the order of 1 T and the maximum gradients are generally greater than 100 T/m, which is desirable to facilitate capture of targeted cells. After a cell mixture is applied to the system, accretion of cellular deposit can form inside a stack of the ferromagnetic spheres in places of the strongest fields and gradients, at points of contact between the beads. The open spaces between the spheres are intended to provide uninterrupted fluid contact across the packed sphere column, necessary for elution of the un-retained, non-magnetic material. The accretion, however, can frequency lead to overloading of the separator due to plugging by the accumulated cellular material in the interstitial spaces of the column as well as trapping nontargeted cells in a targeted cell deposition cake.

Further, the cell separator system 100 is configured to maintain the magnetic and hydrodynamic forces on the cells within a desired range, as discussed above. To obtain high recovery of nontargeted cells, this requirement of no non-specific attachment of cells in the region of magnetic force needs to be extended to any surface within the magnetic cell separation system or support equipment (pump, tubing, containers, etc). As shown previously, optimal cell separation in an annular flow channel occurs when the ratio of the magnetic pressure to the fluid pressure is at least 10. Given that to prevent non-specific binding of cells to a surface a shear stress within all portions of the system should be maintained between 0.01 N/m² and 0.5 N/m², the magnetic pressure in the magnetic separation zone should be at least 0.1 N/m². A number of system parameters can be selected to maintain these optimal ranges, including the magnetic susceptibility of the targeted cells, the flow rate, the field strength of the pole pieces 104-107, the inner and outer diameters of the annular flow channel, and the diameter of the exit tube. Accordingly, a very high level of performance can be achieved.

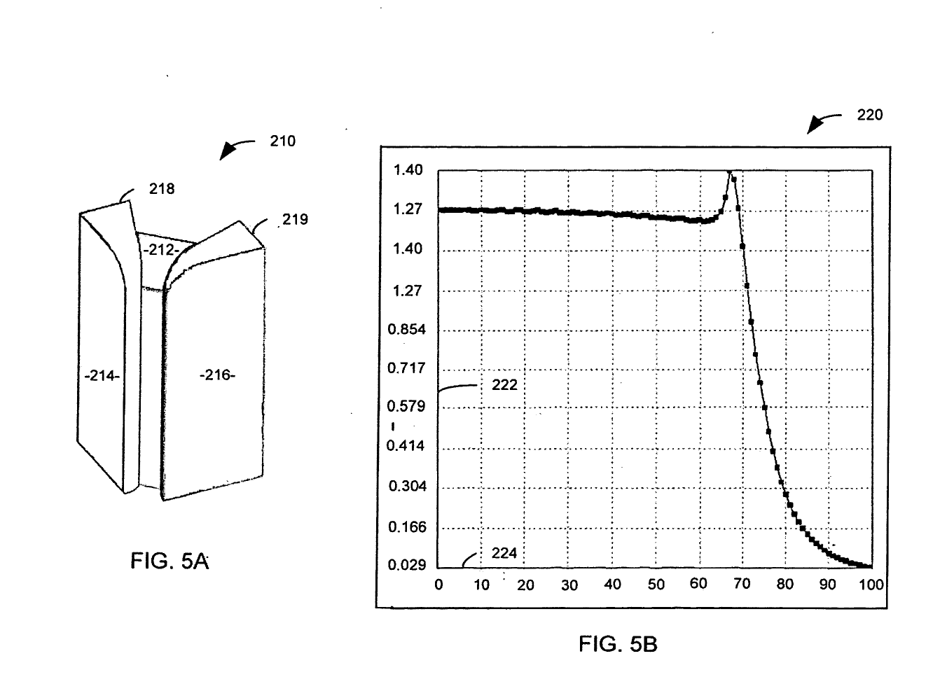

Of equal importance to optimum performance of the separator system is the design of the magnet assembly 104-107, which is configured to eliminate sharp variations in the magnetic field at the entrance. FIG. 5A illustrates one implementation of a pole piece and magnet assembly 210 in accordance with an aspect of the present invention. It will be appreciated that multiple pole pieces and magnet assemblies can be used in a given separator system to produce a substantially uniform magnetic field. For example, the assembly depicted in FIG. 5A can be one of four quarter portions arranged in a circular manner such that sides of assemblies abut each other.

As depicted in FIG. 5A, the pole piece and magnet assembly 210 comprises a magnetic brick 212 positioned between two ferrous pole pieces 214 and 216. In the illustrated example, the ferrous pole pieces 214 and 216 are fabricated from carbon steel. The magnetic brick 212 can be a neodymium-iron-boron magnet. The pole pieces 214 and 216, which direct magnetic flux from the magnet 212, have rounded pole tips 218 and 219 which gradually decrease in distance from a center portion of the assembly 210 where a flow channel of a magnetic separator would be located. The magnetic brick 212 is configured to be shorter than the pole pieces 214 and 216 such that the magnetic brick extends only to the point when the rounded portion of the pole pieces has ended.

It will be appreciated that the configuration of the rounded pole tips 218 and 219 allow for the magnetic field produced by the magnet 212 to be introduced gradually to the center portion of the assembly 210. Specifically, the illustrated assembly 210, used in combination with one or more other assemblies to encompass an associated flow channel produces a magnetic force operating in an outward, radial direction that increases gradually from the entrance into the assembly to a maximal value midway down the axial direction. As a result, spikes that could be caused by a fringing effect associated with the sudden introduction of the targeted cell mixture into the magnetic field at the flow channel inlet close to the proximal (upstream) edge of the pole pieces are avoided, which prevents the accumulation of large deposits of cells at the beginning of the magnet and allows for a gradual deposition of cells down the walls of the separation channel. FIG. 5B illustrates a chart 220 of the magnetic field, represented in units of tesla in the y-axis 222, applied along the length of a flow channel, represented in millimeters from the magnet center on an x-axis 224, by a magnet assembly comprised of four of the magnetic pole pieces and magnet assemblies illustrated in FIG. 5A.

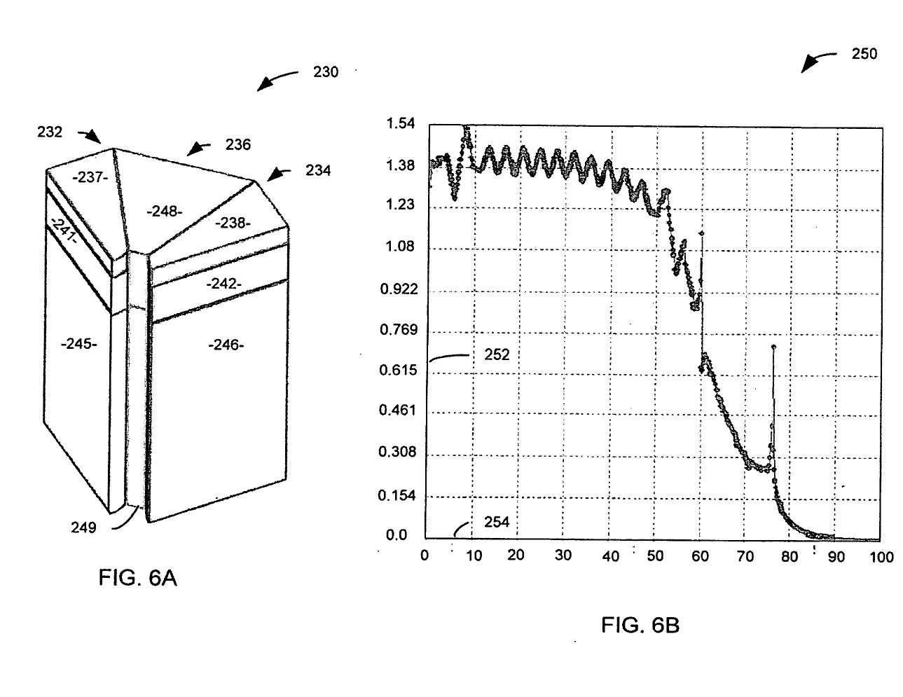

FIG. 6A illustrates a second implementation of a pole piece and magnet assembly 230 in accordance with an aspect of the present invention. In contrast to the shaped pole pieces illustrated in FIG. 5A, the magnet assembly depicted in FIG. 6A regulates the introduction of the magnetic field by having non-rounded pole pieces 232 and 234 but having both the magnet 236 and pole pieces 232 and 234 comprise a plurality of materials having different magnetic properties. Specifically, on the proximal (upstream) edge of each pole piece 232 and 234, is a first layer 237 and 238 of pure nickel, followed by a thicker layer 241 and 242 of cast iron. A final layer 245 and 246 of each pole piece 232 and 234 is made of carbon steel Likewise, the magnet 236 comprises a first layer 248 of aluminum on its proximal (upstream) edge, followed by a second layer 249 of neodymium-iron-boron magnetic bricks.

By selecting materials having desired magnetic properties, the layered approach to the magnet 236 and the pole pieces 232 and 234 illustrated in FIG. 6A allows the magnetic field to be introduced gradually to an associated flow channel. FIG. 6B illustrates a chart 250 of the magnetic field, represented in units of tesla in the y-axis 252, applied along the length of a flow channel, represented in millimeters from the magnet center on an x-axis, 254 by a magnet assembly comprised of four of the magnetic pole pieces and magnet assemblies illustrated in FIG. 6A.

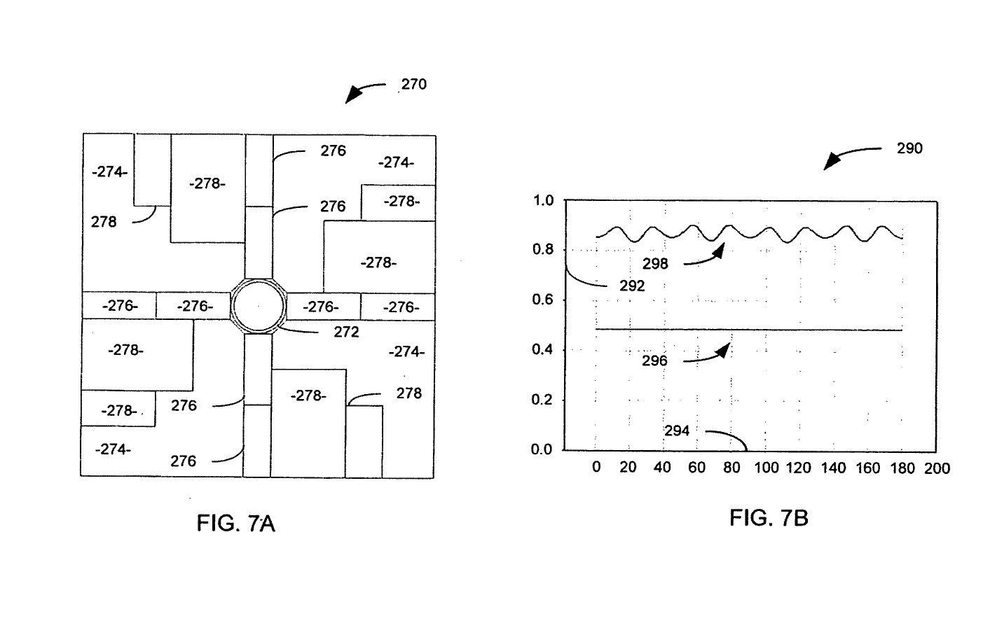

FIG. 7A illustrates a top down view of one implementation of a magnet assembly 270 positioned around an associated flow channel 272 in accordance with an aspect of the present invention. It will be appreciated that either of the designs presented in FIGS. 5A and 6A can be used in combination with the illustrated magnet assembly, specifically with respect to the composition of the upstream portion of the magnet and/or poles, to provide a gradual increase in the magnet assembly. The magnet assembly 270 of FIG. 7A provides a distinctive arrangement of magnetic bricks and pole pieces that produce an angularly symmetric magnetic field within the flow channel 272.

The magnetic assembly 270 comprises a set of magnetically permeable pole pieces 274 that support the magnetic bricks 276 and 278 and provide a path of higher magnetic permeability for the magnetic flux produced by the magnets. In the illustrated implementation, a set of four steel pole pieces 274 are used. A first set of magnetic bricks 274, for example, comprised from Neodymium magnets, are positioned along each of four sides of the flow channel 272 to provide a main source of magnetic field strength to the flow channel. To increase the uniformity of the magnetic field at the flow channel 272, a second, weaker set of magnetic bricks 278, for example, Ceramic-5 magnets, can be positioned around the pole pieces 274 to provide supplementary field strength at the gaps between the first set of magnetic bricks 276. Accordingly, the illustrated magnetic assembly 270 generates a substantially uniform magnetic field for a given radial position within the flow channel 272 using commercially available or easily fabricated rectangular magnetic bricks and pole pieces.

FIG. 7B illustrates a chart 290 of magnetic field, represented in units of tesla in the y-axis 292, applied directionally around an annular flow channel encompassed by the magnet assembly illustrated in FIG. 7A. The x-axis 294 represents the angular direction around the annular flow channel in degrees. A first graphic 296 represents the applied magnetic field at an inner wall of the annular flow channel, while a second graphic 298 represents the applied magnetic field at an outer wall of the annular flow channel. It will be appreciated that, for a constant radial distance within the annular flow channel, the applied magnetic field is substantially constant, allowing for a uniform distribution of cell caking around the circumference of the annular flow channel.

The gradual increase of the field strength along the entire length of the axial flow direction of the cell suspension inside the magnet, as mentioned previously, is a distinct feature of this invention. This feature is designed for a maximum cell capture from a magnetically distributed cell population. For example, a magnetic cell tagging process, such as a tagging process using magnetic nanoparticle-antibody conjugates against cell surface antigens, produces distributed effective cell susceptibility across the targeted cell population because of the inherently distributive nature of the antibody—antigen binding. The average number of the bound magnetic tags, and therefore the average magnetic susceptibility of the targeted cell population, is governed by the law of mass action applied to the antibody—antigen reaction. This creates a distribution in cell population: a fraction of the targeted cell population will have a number of bound magnetic tags that is lower than the mean and a fraction will have a number of bound magnetic higher than the mean. Typically, the number of such fractions is distributed normally around the mean, forming what is known as the bell-shaped curve (Gaussian distribution). This implies that the magnetic cell tagging will produce a majority fraction with a characteristic average susceptibility, and smaller fractions with lower or higher susceptibilities than the average.

The gradual increase of the magnetic field strength along the fluid path of the cell suspension ensures a desirable, uniform thickness of the captured cell cake on the wall of the annular channel separated from the magnetically distributed cell suspension because the highly magnetic cell fraction will deposit at the proximal (upstream) portion of the channel and the weakly magnetic cell fraction will deposit at the distal (downstream) portion of the channel inside the magnet assembly. In other words, in the frame of reference of the cell traveling down the annular channel inside the pole piece assembly, the cell is exposed to the gradually increasing magnetic field pressure produced by the pole piece assembly, p_{mag}, which is the function of its effective susceptibility, χ_{eff} (See Equation 2).

The presence of a constant fluid shear stress at the surface (either of a channel wall or the surface of the forming cell deposition cake) prevents the cell from the deposition until such point in its travel where the magnetic pressure, p_{mag}, significantly exceeds the fluid shear stress, SS (See Equation 11), where the cell becomes captured. The less magnetically susceptible cells will travel farther along the annular channel and therefore deposit downstream from the more magnetically cells, leading to a more uniform cell deposition cake and a greater channel capacity when compared with the magnet producing a constant magnetic field along the axial coordinate. By matching the parameters SS and p_{mag}, as discussed above, one ensures the capture of the entire targeted cell population with the distributed magnetic susceptibility, preventing the local overload of the highly magnetic cell fraction at the upstream lip of the magnet assembly and the loss of the weakly magnetic cell fraction due excessively high SS.

In view of the foregoing structural and functional features described above, methodology in accordance with various aspects of the present invention will be better appreciated with reference to FIG. 8. While, for purposes of simplicity of explanation, the methodology of FIG. 8 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect the present invention.

FIG. 8 illustrates a method 300 for separating a first group of cells from a mixture of at least first and second groups of cells in a suspension in accordance with an aspect of the present invention. At step 302, a first group of cells is labeled to produce a plurality of targeted cells having a selected magnetic susceptibility, resulting in a plurality of the magnetic pressure exerted on the targeted cells For example, the cells can be immunologically tagged with antibodies that have been covalently linked to a particle having desired magnetic properties. Within limits, the particle selected can influence the magnetic susceptibility of the targeted cells, and the magnetic pressure exerted on the cell, allowing for limited manipulation of this property.

At step 304, an optimal volumetric flow for a flow channel is determined based on the mean and distribution of the magnetic pressure operating on the targeted cell population, a geometry of the flow channel, and a magnitude of a magnetic field associated with the flow channel as to maximize a percentage of the first group of cells that adhere to a surface within the magnetic field, as discussed above. For example, the optimal volumetric flow can be determined from a range of optimal shear stresses, based on the geometry of the system and the viscosity of the fluid. Similarly, the flow can be selected such that a desired relationship between the magnetic forces and the fluid forces is maintained. At step 306, the suspension is pumped through the flow channel and its associated magnetic field at the determined optimal volumetric flow rate.

It will be understood that the above description of the present invention is susceptible to various modifications, changes and adaptations, and the same are intended to be comprehended within the meaning and range of equivalents of the appended claims. The presently disclosed embodiments are considered in all respects to be illustrative, and not restrictive. The scope of the invention is indicated by the appended claims, rather than the foregoing description, and all changes that come within the meaning and range of equivalence thereof are intended to be embraced therein.

Having described the invention, the following is claimed:

1. A method for separating a first group of cells from a mixture of at least first and second groups of cells in a suspension, comprising:
magnetically labeling the first group of cells to produce a plurality of labeled cells having an selected magnetic susceptibility;
determining an optimal volumetric flow for a given flow channel from a distribution of the magnetic susceptibility of the plurality of labeled cells, a strength of a magnetic field applied to the flow channel, and a geometry of the flow channel as to maximize a percentage of the first group of cells that adhere to a surface within the magnetic field; and
pumping the suspension through the flow channel at the determined optimal volumetric flow rate.

2. The method of claim 1, wherein determining the optimal volumetric flow rate comprises selecting a volumetric flow rate as to maintain a ratio of a maximum magnetic pressure acting on the cells to the hydrodynamic shear stress acting on the labeled cells within an optimal range.

3. The method of claim 2, wherein selecting the volumetric flow rate comprises selecting the volumetric flow rate as to maintain the ratio of the maximum magnetic pressure acting on the labeled cells to the hydrodynamic shear stress acting on the labeled cells at a value above ten.

4. The method of claim 1, wherein determining the optimal volumetric flow rate comprises selecting a volumetric flow rate as to maintain a ratio of a maximum magnetic pressure acting on the second group of cells to the hydrodynamic shear stress on the second group of cells within an optimal range.

5. The method of claim 4, wherein comprises selecting the volumetric flow rate comprises selecting the volumetric flow rate as to maintain the ratio of the maximum magnetic pressure acting on the second group of cells to the hydrodynamic shear stress acting on the second group of cells to be a value less than one-tenth.

6. The method of claim 1, wherein determining the optimal volumetric flow rate comprises selecting a volumetric flow rate as to maintain a shear stress within an optimal range at all internal surfaces of the flow channel.

7. The method of claim 6, wherein pumping the suspension through the magnetic field comprises pumping the suspension through an annular flow channel having an inner surface with a first radius and an outer surface with a second radius, and the volumetric flow rate being selected as a function of a desired value for the shear stress, the first radius, and the second radius.

8. The method of claim 6, the optimal range for the shear stress comprising values between 0.01 N/m² and 0.5 N/m².

9. The method of claim 6, wherein selecting the volumetric flow rate comprises selecting the volumetric flow rate such that the maximum value of the local energy dissipation rate at any location in the flow channel of the flow channel does not exceed five hundred W/m³.

10. The method of claim 1, wherein the magnetic field is configured to increase gradually along an axis of flow to a maximum value.

11. The method of claim 10, wherein configuring the magnetic field comprises positioning at least one magnet pole piece adjacent to the flow channel, the at least one magnet pole piece being shaped to produce a magnetic field that increases gradually along an axis of flow.

12. The method of claim 10, wherein the configuring the magnetic field comprises positioning at least one magnet pole piece adjacent to the flow channel, a given magnetic pole piece comprises a plurality of layers of magnetic material having varying magnetic field strengths.

13. A system that separates a first group of cells from a mixture of at least first and second groups of cells in a suspending fluid, comprising:
an annular flow channel;
at least one magnetic element positioned around the exterior of the annular flow channel as to provide a radially symmetric magnetic field around at least a portion of the annular flow channel, the at least one magnetic element being configured to provide a gradually increasing magnetic field along an axis of flow as to limit a maximum magnetic gradient within the annular flow channel; and
a pump, operatively connected to a terminal end of the annular flow channel, that forces the suspending fluid through the annular flow channel.

14. The system of claim 13, wherein the at least one magnetic element comprises a plurality of pole pieces positioned around the annular flow channel.

15. The system of claim 14, wherein each of the plurality of pole pieces comprises a plurality of magnetic plates, the magnetic plates comprising a plurality of different materials, having different magnetic properties, and being arranged as to produce the gradually increasing magnetic field.

16. The system of claim 13, the pump being connected to the terminal end of the annular flow channel via an exit line.

17. The system of claim 13, the annular flow channel having an inner radius, r, and outer radius, R, and the pump being operative to produce a volumetric flow rate, Q, such that for a fluid having a viscosity, η:
$$0.01 \leq η ( \frac{4 Q}{( 1 - {( \frac{r}{R} )}^{4} - \frac{{( 1 - {( \frac{r}{R} )}^{2} )}^{2}}{ln ( \frac{R}{r} )} ) π {R}^{3}} ( \frac{r}{R} - \frac{1 - {( \frac{r}{R} )}^{2}}{ln ( \frac{R}{r} )} \frac{R}{r} ) ) \leq 0.5 .$$

18. The system of claim 13, wherein the pump has an associated volumetric flow rate which is selected to maintain a ratio of a magnetic pressure introduced by the at least one magnetic element on the first group of cells to a fluid shear stress created by the pump at a value greater than ten.

19. A system that separates a first group of cells from a mixture of at least first and second groups of cells in a suspending fluid, comprising:
an annular flow channel;
at least one magnetic element, the at least one magnetic element substantially encompassing the annular flow channel, such that a magnetic field is provided through at least a selected portion of the annular flow channel; and
a pump, operatively connected to a terminal end of the annular flow channel, that forces the suspending fluid through the annular flow channel at a flow rate, the flow rate and a magnitude of the magnetic field being selected as to maintain a ratio of magnetic pressure created by the magnetic field on the first group of cells to a shear stress generated by the pump above a threshold value.

20. The system of claim 19, wherein the at least one magnetic element comprises a plurality of pole pieces positioned around the annular flow channel, each of the plurality of pole pieces comprising a plurality of magnetic plates, the magnetic plates being formed from a plurality of different materials, having different magnetic properties, and being arranged as to produce a gradually increasing magnetic field along an axis of flow as to limit a maximum magnetic gradient at an upstream lip of the annular flow channel.

21. The system of claim 20, wherein at least one magnetic element comprises at least one magnetic pole piece, a gradually increasing magnetic field along an axis of flow as to limit a maximum magnetic gradient within the annular flow channel is produced by suitably shaping the plurality of pole pieces.

22. The system of claim 19, wherein the at least one magnetic element comprises a plurality of pole pieces positioned around the annular flow channel, each of the plurality of pole pieces comprising a plurality of magnetic plates, the magnetic plates being formed from a plurality of different materials, having different magnetic properties, and being arranged as to produce a gradually increasing magnetic field along an axis of flow as to produce a maximum magnetic gradient at a downstream lip of the annular flow channel.

23. The system of claim 22, wherein the plurality of pole pieces are suitably shaped to provide a gradually increasing magnetic field along an axis of flow as to produce a maximum magnetic gradient at the distal (downstream) lip of the annular flow channel.

24. The system of claim 19, the annular flow channel having an inner radius, r, and outer radius, R, the suspending fluid having a viscosity, η and the flow rate, Q, being selected such that:
$$0.01 \leq η ( \frac{4 Q}{( 1 - {( \frac{r}{R} )}^{4} - \frac{{( 1 - {( \frac{r}{R} )}^{2} )}^{2}}{ln ( \frac{R}{r} )} ) π {R}^{3}} ( \frac{r}{R} - \frac{1 - {( \frac{r}{R} )}^{2}}{ln ( \frac{R}{r} )} \frac{R}{r} ) ) \leq 0.5 .$$

25. The system of claim 19, wherein the threshold value is ten.

26. The system of claim 19, the annular flow channel having a solid inner core that provides support for the annular flow channel.
